(19) United States
(12) Patent Application Publication
(10) Pub. No.: US 2015/0282692 A1
(43) Pub. Date: Oct. 8, 2015

(54) ELECTROMAGNETIC ACTUATOR FOR A SURGICAL INSTRUMENT

(71) Applicant: OLYMPUS WINTER & IBE GMBH, Hamburg (DE)

(72) Inventors: Martin WIETERS, Hamburg (DE); Andreas NOACK, Drage (DE)

(73) Assignee: OLYMPUS WINTER & IBE GMBH, Hamburg (DE)

(21) Appl. No.: 14/742,803

(22) Filed: Jun. 18, 2015

Related U.S. Application Data
(63) Continuation of application No. PCT/EP2013/003622, filed on Dec. 2, 2013.

(30) Foreign Application Priority Data
Dec. 21, 2012 (DE) 102012224179.5

(51) Int. Cl. A61B 1/00; H01F 41/02; H01F 7/126; H01F 7/02; H01F 7/121

(52) U.S. Cl. CPC A61B 1/00068; H01F 7/0231; H01F 7/0273; H01F 7/121; H01F 7/126; A61B 1/00096; A61B 1/00183; A61B 1/00188; H01F 41/0266

(57) ABSTRACT

An electromagnetic actuator for a medical instrument. The electromagnetic actuator including: a stator; a movable element, which at least partially comprises one or more of a paramagnetic and ferromagnetic material and which is movable from a first position to a second position by the application of an electromagnetic field, and a tube movably supporting the movable element in such a way that the movable element is longitudinally movable, wherein the tube comprises a ferromagnetic material.

### CROSS-REFERENCE TO RELATED APPLICATION

The present application is a continuation of PCT/EP2013/003622 filed on Dec. 2, 2013, which is based upon and claims the benefit to DE 10 2012 224 179.5 filed on Dec. 21, 2012, the entire contents of each of which are incorporated herein by reference.

### BACKGROUND

1. Field

The present application relates to an electromagnetic actuator for a surgical or medical instrument, in particular endoscope, wherein the actuator comprises a stator and a movable element, which at least partially comprises a paramagnetic and/or ferromagnetic material and which can be moved from a first position to a second position by the application of an electromagnetic field, wherein the movable element is supported in a tube in such a way that the movable element is longitudinally movable. The application further relates to a method for producing a tube.

2. Prior Art

An endoscope with a distally arranged objective is known from DE 196 18 355 C2, the image of which is forwarded to the proximal end by an image forwarder and that has at least one optical element, such as a lens group, which is shiftable in the direction of the optical axis for focussing and/or for changing the focal length by a microthruster, wherein the microthruster has at least one rotationally symmetrical axially movable sleeve which surrounds and receives the lenses or respectively the optical element of the movable lens group and wherein the sleeve is made of a permanently magnetic material and is movable in a magnetic field which is generated by a coil arrangement. In order to move and to hold the sleeve, an electromagnetic field is generated continuously.

An endoscope with a distally radiating illumination device for a visceral cavity part to be observed and an image conductor is known from DE 1 253 407 B, which captures the illuminated image via an objective that is adjustable in the axial direction and directs it to an ocular or a camera, wherein the objective is adjustable for at least two image sharpness settings from one position into another position with respect to the distal end of an image conductor through electromagnetic manipulation of an objective mount serving as an anchor. At least one of the two positions is hereby evoked by a permanently present electromagnetic field and the other position by the effect of a spring.

DE 10 2011 006 814 A1 discloses an electromagnetic actuator for a surgical or medical instrument (hereinafter collectively referred to as a medical instrument), wherein the actuator comprises a stator and a movable element which is at least partially composed of a paramagnetic or ferromagnetic material and which can be moved from a first position to a second position by the application of an electromagnetic field. Moreover, a tube is provided, in which the movable element is supported in such a way that the movable element is longitudinally movable.

### SUMMARY

An object is to specify an electromagnetic actuator, by means of which a powerless holding of the movable element in defined positions is possible, wherein the moving of the movable element of the actuator with low power and with good efficiency should be enabled.

The object can be solved by an electromagnetic actuator for a surgical or medical instrument, in particular an endoscope, wherein the actuator comprises a stator and a movable element which at least partially comprises a paramagnetic and/or ferromagnetic material and which can be moved from a first position to a second position by the application of an electromagnetic field, wherein the movable element is supported in a tube in such a way that the movable element is longitudinally movable, wherein the tube comprises a ferromagnetic material.

Through use of a tube that comprises a ferromagnetic material, the permeability is increased in comparison to an air gap or in comparison to a tube, which, as in the state of the art, does not contain a ferromagnetic material. The holding and switching forces of the electromagnetic actuator are hereby changed in comparison to the state of the art. In particular, by increasing the permeability, the magnetic circuit around a coil provided for generating the electromagnetic field upon activation of the coil is closed better, whereby the electromagnetic field generated by the coil and in particular the magnetic flux is increased. The switching force is hereby increased and in particular the efficiency of the electromagnetic actuator is increased. The ferromagnetic material can be a ferrimagnetic material.

The permeability of the tube can lie at least partially between 1.2 and 200, in particular between 2 and 200, more particularly between 5 and 20. A range of 2 to 100 could also sensibly be provided.

The permeability of the tube can lie at least in sections in a range, the lower limit of which is 1.2. The lower limit can be 2. Furthermore, the lower limit can be 3, 4 or 5. The upper limit of the permeability of the tube, which can be present at least in sections, can be 200, in particular 100. In particular, the upper limit can be 40, 30, 25 or 12. Ranges for the permeability can be more particularly from 1.2 to 100, 1.2 to 40, 2 to 30, 4 to 25 or 5 to 12.

The material of the tube or of sections of the tube can be a metal alloy, which has a corresponding permeability. It can also be a ferrite material, for example a nickel-iron compound. Moreover, the tube can contain a plastic filled with ferromagnetic particles, since this variant is easy to produce and also has less resistance compared to the rotor or respectively compared to the movable element so that a movement of the movable element is already possible with little force. The permeability can be distributed evenly over the entire tube.

The tube can have areas in the axial direction where the permeability is different with respect to each other. The magnetic flux lines can hereby be set in the desired manner. If at least one area adjacent to a middle area of the tube has a higher permeability than the middle area, a magnetic short circuit is efficiently prevented, whereby the efficiency is considerably increased.

At least one area of the tube can have an anisotropic permeability.

It is hereby prevented in particular that the tube magnetically short circuits a magnetic south pole and a magnetic north pole of a magnet which is arranged on the tube or respectively near the tube. In particular, an embodiment in which the magnetic flux in the radial direction of the tube is higher than in the axial direction is possible.

The tube can include, in the circumferential direction, areas, the permeability of which is different than the respective adjacent area in the circumferential direction. The movable element can hereby be prompted not to rotate in the tube or to only rotate slightly in the event of an executed longitudinal movement. For example, two, four or more areas can be arranged next to each other in the circumferential direction, wherein the permeability of the adjacent areas is different with respect to each other.

The electromagnetic actuator can be further developed in that the movable element is or will be held in the first position by a permanent magnetic field and, after movement into the second position, is or will be held in the second position by a permanent magnetic field.

It is possible through use of a permanent magnetic field to hold the movable element, in particular in succession, in a powerless manner both in the first as well as in the second position so that no further power needs to be brought into the system.

An embodiment, in which the stator comprises two permanent magnets, which are oppositely poled, is also possible. In such embodiment, oppositely poled means that the poles of the two permanent magnets arranged with respect to each other repel each other, i.e. the same poles are adjacent to each other. It is hereby particularly easy to enable a powerless holding of the movable element in the first and/or the second position. The movable element can contain no permanent magnet but rather can consist exclusively of a paramagnetic and/or a ferromagnetic material and, if applicable, additionally a non-magnetic material, wherein the ferromagnetic material can be due to the greater magnetic-field-strengthening effect.

A coil, which can be arranged between the permanent magnets, can be provided in order to generate the electromagnetic field. This arrangement makes it possible to move the movable element even with a relatively small electromagnetic field. During the moving or respectively switching of the electromagnetic actuator, the permanent magnetic field of the two permanent magnets and the electromagnetic field of the coil work together. It is hereby enabled that the permanent magnets are not demagnetized by the electromagnetic field.

Two stops that define the first and the second position can be provided. Through the stops, the movable element comes into the corresponding end positions or intermediate positions, over which the movable element cannot pass beyond. Upon placement of the movable element on a stop, an in particular not disappearing force can act on the movable element in the direction of the stop. The movable element can be pulled in the direction of a metastable position, into which the movable element can, however, not fully reach due to the stops. In this respect, a magnetic force acts in the respective positions, i.e. in the first position, in the case in which the movable element fits in the first position, and also in the case in which the movable element fits in the second position, in the direction of the respective stop so that the movable element is held in a defined manner on the stop. A very defined position thereby results.

Instead of the stop, it would also be possible to not provide a stop and to enable a first or respectively second position in the area of an energetic minimum of the cooperation of the permanent magnetic field through the permanent magnets and of the material of the movable element.

If a paramagnetic and/or ferromagnetic material is arranged between the permanent magnets of the stator, a particularly small power is sufficient for the electromagnetic field in order to enable a movement of the movable element from a first position into a second position or vice versa. The paramagnetic and/or ferromagnetic material is hereby in particular part of the stator.

The coil can be surrounded towards the outside by the permanent magnets and the paramagnetic and/or ferromagnetic material, in particular of the stator.

Through the arrangement of the paramagnetic and/or ferromagnetic material, both in the movable element as well as in the stator, a magnetic flux guidance is generated for the coil, whereby high magnetic fields and thus high power density can be achieved already with small flows or fluxes through the coil.

The longitudinal movement of the movable element can be along the longitudinal axis of the tube. The tube can be cylindrical. A magnetic field symmetrical, in particular rotationally symmetrical, around the longitudinal axis can be generated. Hereby and in particular through the measure that the movable element, the coil, the tube and/or the permanent magnets are annular in cross-section, and namely in particular in cross-section transversely to the longitudinal axis, constant forces act on the movable element so that movement is possible with little effort. For the movement procedure of the movable element or respectively the switching procedure from a first position into a second position or vice versa, a short electrical switching impulse through the coil of less than 100 milliseconds and less than 500 milliamperes suffices.

A surgical or medical instrument, in particular an endoscope, can be provided with the electromagnetic actuator according to the present application.

Furthermore, the object is solved by a method for producing a tube, in particular for use in an electromagnetic actuator, with the following method steps:

provision of a casting mould, in which at least one magnet is arranged,

introduction of a casting compound into the casting mould, wherein at least in the area, in which the at least one magnet is arranged, the casting compound has ferromagnetic particles and

hardening of the casting compound for formation of a stable tube.

The hardening of the casting compound can take place in the casting mould so that the ferromagnetic particles retain their alignment after their alignment provided by the magnetic field even after removal of the tube from the casting mould. In particular, a complete hardening in the casting mould can be provided. At least two areas in the casting mould can be provided, wherein, in a first area, a casting compound with ferromagnetic particles is introduced and, in a second area, a casting compound without ferromagnetic particles is introduced. A magnetic field aligning the ferromagnetic particles can be provided in the first area by a magnet arranged in the casting mould. A casting compound can be introduced into the casting mould in a middle area of the casting mould that has no ferromagnetic particles and the casting compound with ferromagnetic particles is introduced into at least two of these areas bordering this middle area.

The ferromagnetic particles can be aspherical and in particular elongated. A type of magnetized needle, which ensures an anisotropic permeability of the tube during operation or respectively after installation of the tube into an electromagnetic actuator thereby results.

### BRIEF DESCRIPTION OF THE DRAWINGS

The embodiments are described below, without restricting the general idea of the invention, using exemplary embodiments with reference to the drawings, whereby we expressly refer to the drawings with regard to all details according to the invention that are not explained in greater detail in the text. In the figures:

FIG. 1 illustrates a schematic, three-dimensional sectional representation through a part of an endoscope with an actuator,

FIG. 2 illustrates a schematic sectional enlargement from FIG. 1,

FIG. 3 illustrates a schematic sectional representation of another embodiment of an actuator a,

FIG. 4 illustrates a schematic sectional representation of the embodiment from FIG. 3 with a schematic flux representation,

FIG. 5 illustrates a schematic sectional representation of the embodiment from FIG. 3 with a schematic flux representation,

FIG. 6 illustrates a schematic sectional representation of a part of an actuator,

FIG. 7 illustrates a schematic top view of a tube,

FIG. 8 illustrates a schematic sectional representation of a casting mould,

FIG. 9 illustrates a schematic representation of a tube, and

FIG. 10 illustrates a diagram of the force, plotted over a permeability.

### DETAILED DESCRIPTION

In the following figures, the same or similar types of elements or respectively corresponding parts are provided with the same reference numbers so that a corresponding re-introduction can be omitted.

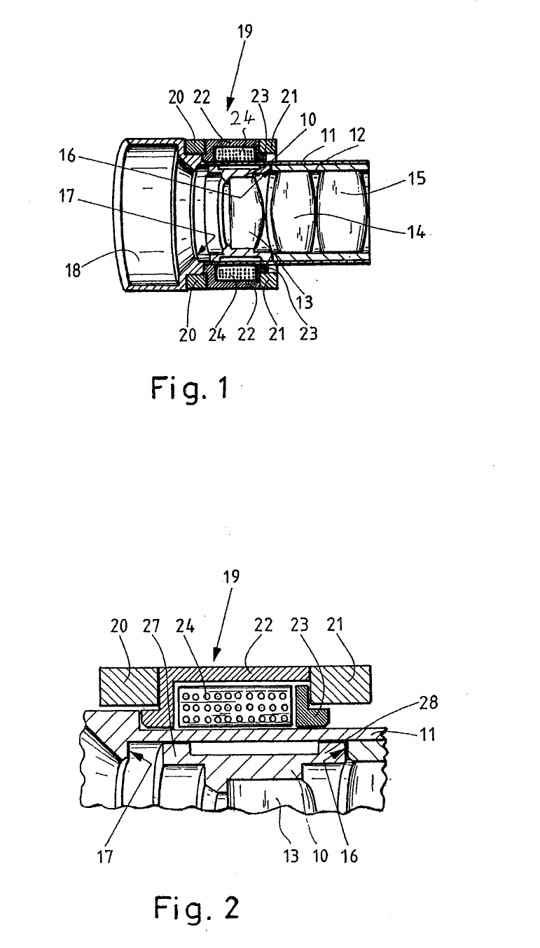

FIG. 1 shows a schematic, three-dimensional sectional representation through a part of an endoscope with an actuator. The actuator can be arranged in a shaft (not shown) of an endoscope. In FIG. 1, the shaft of the endoscope would be arranged coaxially around the actuator, namely with a diameter which is slightly larger than the outer diameter of the distal end 18 of the sliding tube 11.

The sliding tube 11 contains a ferromagnetic material and serves as a radial guide of the movable element 10. The movable element 10 can have, for example, a lens 13, which is part of an objective, which also has lenses 14 and 15, which are inserted in a locked holding element 12 and are correspondingly held. The locked holding element 12 is locked or respectively attached in the sliding tube 11 and defines a stop 16. The additional stop 17 to the distal end is also defined by the sliding tube 11 through a collar inwards. This exemplary embodiment according to FIG. 1 has a rotationally symmetrical structure, in which an axially movable element 10 is provided. The axially moveable element 10 can be moved from a proximal position, as shown in FIG. 1, to the left towards the stop 17 into a distal position. The moveable element 10 is designed as a type of sleeve which is made in particular of a magnetically soft material, such as a ferromagnetic material or respectively includes such material.

Besides comprising ferromagnetic and/or paramagnetic material, the movable element 10 can also have a friction-reducing coating on a surface which is arranged towards the inside wall of the sliding tube 11.

The tube 11 or respectively sliding tube thus has a permeability which is greater than 1 and in particular lies in a range between 1.5 and 200, more particularly between 2 and 100, and even more particularly between 5 and 20. The tube can be made of a material or contain a material that has a corresponding alloy which has this permeability. A ceramic can also be provided with such a permeability or a ceramic, into which particles, for example ferromagnetic particles, are introduced. Correspondingly, a plastic can also be provided as sliding tube 11 or respectively tube 11, into which the ferromagnetic particles are introduced.

FIG. 2 shows a sectional enlargement from FIG. 1, in which the shape of the respective elements can be clearly identified. The movable element 10 has a distal pole shoe 27 and a proximal pole shoe 28. These work together with the magnetic field and the permanent magnets 20 and 21, which are designed as rings and are arranged rotationally symmetrically around the longitudinal axis of the electromagnetic actuator. A first intermediate part 22 and a second intermediate part 23 made of paramagnetic or ferromagnetic material, which are also designed with pole shoes or as pole shoes, are provided between the permanent magnets 20 and 21. The first intermediate part 22 and the second intermediate part 23 can also be one-piece, i.e. form a single intermediate part. Furthermore, a coil 24 is provided which is surrounded to the outside by the first intermediate part 22 and the second intermediate part 23 and is surrounded to the inside except for the interruption by the sliding tube 11 also by paramagnetic and/or ferromagnetic material of the moveable element 10. A very strong strengthening of the electromagnetic field is thereby achieved. The stator 19 of the electromagnetic actuator consists mainly of the two permanent magnetic rings 20 and 21, the two intermediate parts 22 and 23 and of the coil 24.

The material, from which the movable element 10 can be made or respectively that it has, can be for example St-37 or C-45k. The outer contour of the movable element represents a double anchor. Two pole shoes, namely a distal pole shoe 27 and a proximal pole shoe 28, hereby develop. Moreover, the outsides of the pole shoes serve as sliding surfaces for the sliding pairing between the sliding tube 11 and the movable element 10. The inner contour of the movable element can be axially symmetrical. However, it is possible to deviate from the symmetry to a certain extent in order to integrate for example a shoulder for the installation of a lens 13. The movable element can be designed in black matte.

The stator 19 mainly contains two similar permanent magnets which have the same material or respectively the same magnetic and magnetization strength and correspondingly the same dimensions. Furthermore, a coil 24 as well as two ferromagnetic components or respectively intermediate parts 22 and 23 which serve as magnetic flux guidance for strengthening and focussing of magnetic fields, are provided. The intermediate parts 22 and 23 are horseshoe-shaped in cross-section longitudinally through the stator and realized in a pole-shoe-like symmetrical design. Both, the movable element 10 as well as the stator 19 can be built axially symmetrically. The permanent magnets 20 and 21 are oppositely poled or respectively installed in an engaged manner.

The electromagnetic actuator can be present in four different states. The first state is the state shown in FIGS. 1 and 2, in which the movable element 10 is located in the stable proximal position. The resulting force of the permanent magnets thereby acts on the movable element against the proximal stop 16. Furthermore, the movable element can be located in a stable distal position which is not shown in FIGS. 1 and 2. The resulting force of the permanent magnets then acts on the movable element 10 against the distal stop 17.

The third state is where the actuator moves the movable element out of the distal position. The resulting force of the coil and the permanent magnets then moves the movable element 10 in the proximal direction. Conversely, the fourth state is defined, in which the actuator moves the movable element 10 out of the proximal position. The resulting force of the coil and the permanent magnets is thereby such that the movable element 10 is moved in the distal direction.

The functionality is explained in greater detail below.

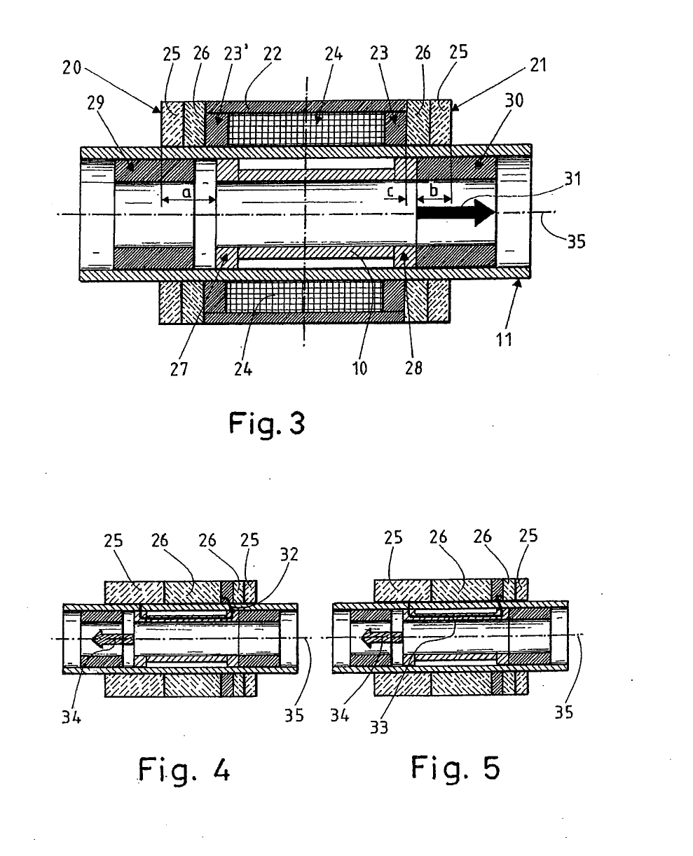

Schematic sectional representations through an electromagnetic actuator are shown in FIGS. 3 to 5, wherein the respective elements and characteristics are indicated schematically. In FIG. 3, the coil 24 is powerless, i.e. it does not create a magnetic field. As in FIGS. 1 and 2, the stator correspondingly contains intermediate parts 22, 23 and 23′, which are designed in a horseshoe-like manner in cross-section, made of a ferromagnetic material. The intermediate parts 22, 23 and 23′ can be produced as one common piece, i.e. as one piece.

Number 25 schematically shows a magnetic south pole and number 26 shows a schematic north pole. Number 22 shows a first intermediate part or respectively component and number 23 and 23′ each show a second intermediate part or respectively component designed as a pole shoe. Correspondingly, the elements 10, 27 and 28, which should represent the ferromagnetic parts of the movable element 10, can also be jointly one-piece. Number 27 indicates the distal pole shoe and 28 the proximal pole shoe.

In this case, the holding forces of the movable element are only generated by the two permanent magnets through a permanent magnetic field.

Through the engaged magnets 20 and 21, the same magnetic pole is located on both pole shoes 23 and 23′ of the stator. The magnetic flux tries to follow the path of the lowest magnetic resistance. Compared to air, the magnetic resistance of the used ferromagnetic material is much lower so that the system on the whole tries to minimize the air gaps. This is called reluctance. The pole shoes, which can be made of magnetically soft or respectively ferromagnetic material, are thereby brought to overlap, whereby a movement or respectively a force is realized.

In order to achieve a holding force in the proximal direction, as indicated in FIG. 3 by the force 31, towards the proximal stop element 30, the following should be given. The proximal pole shoe 28 of the movable element 10 must be positioned closer to the proximal end of the proximal permanent magnet 21 than the distal pole shoe 27 of the movable element to the distal end of the distal permanent magnet 20. Thus, a must be greater than b. Moreover, the proximal pole shoe 28 of the movable element 10 must protrude proximally over the proximal pole shoe 23 of the anchor. Thus, c must be greater than zero. If c were to equal 0, the system would be in the magnetic or respectively in the energetic minimum. There would then no longer be a resulting force 31. A corresponding force in the direction of the energetic minimum would only occur in the case of a movement out of this position. This leads to a non-discrete positioning.

The movable element 10 forms for both magnets 20 and 21 the magnetic return path so that the lowest magnetic resistance or respectively the most energetically beneficial state of the system can be achieved via the movable element 10. Depending on the position of the movable element, i.e. also depending on the position of the stop elements 29 or respectively 30, different holding forces can thus be realized. In the example shown, the electromagnetic actuator is designed such that the position of the movable element 10 on the stop, i.e. for example on the proximal stop element 30, does not correspond with the most energetically beneficial state. The electromagnetic actuator will thereby continue to try to pull the movable element into the position of the lowest resistance, whereby the resulting holding force (reluctance) results.

In order to now move the movable element 10 from the proximal position into the distal position, the coil 24 is supplied with current. A total magnetic field can thereby be generated which generates a force in the distal direction, which is greater than the holding force in the proximal direction. This is shown in FIGS. 4 and 5. The force in the distal direction is specified as moving force 34. By supplying current to the coil 24, a corresponding magnetic field results in the summation of the magnetic field of the distal permanent magnet 20 and of the coil, which is indicated schematically by a magnetic north pole 26 and a magnetic south pole 25 on the left side of FIG. 4 and FIG. 5. The coil ideally generates a magnetic flux which corresponds with the distal permanent magnet 20. The magnetic field is thereby strengthened towards the proximal second intermediate part 23 or respectively stator pole shoe. The distal permanent magnet 20 and the coil form abstractly a large cohesive magnet which has schematically a larger, ideally doubled, field strength than the proximal permanent magnet 21. Corresponding magnetic flows or fluxes 32 and 33, which are shown in FIGS. 4 and 5, respectively, and a corresponding moving force 34 towards the distal end hereby result. Through the cooperation of the three magnetic components (both permanent magnets 20 and 21 and the coil 24), the movable element 10 is moved out of its proximal position towards its distal position.

Through the shown construction, it is not necessary that the magnetic flux of the coil completely extinguishes the magnetic flux of a permanent magnet. The risk of the magnetic field of the coil demagnetizing the permanent magnet is thereby reduced. A very high efficiency is achievable by surrounding the coil with ferromagnetic material. This minimizes the necessary switching current and thus potential heating, which should be avoided in the distal area of an endoscope.

In the case of electromagnetic actuators according to the state of the art, corresponding guides of a movable element are used, for example a guide tube or a tube, which is made for example of stainless steel, a ceramic or plastic and has a permeability μᵣ of 1 or respectively approximately 1 and thus for magnetic fields behaves similar to air. In particular in the case of greatly miniaturized electromagnetic actuators, which can also be called reluctance actuators, it is important to keep the efficiency as high as possible since in the miniaturization the forces decrease to the fourth power. For this, for example, the air gap between the magnets and the movable element could be reduced. However, based on the use of a guide tube or respectively tube, a minimum thickness is required. The air gap thus cannot be reduced infinitely and the efficiency cannot be increased optimally. According to the embodiments disclosed herein, the permeability of the guide tube or respectively of the tube is now increased in order to reduce the “air gap”.

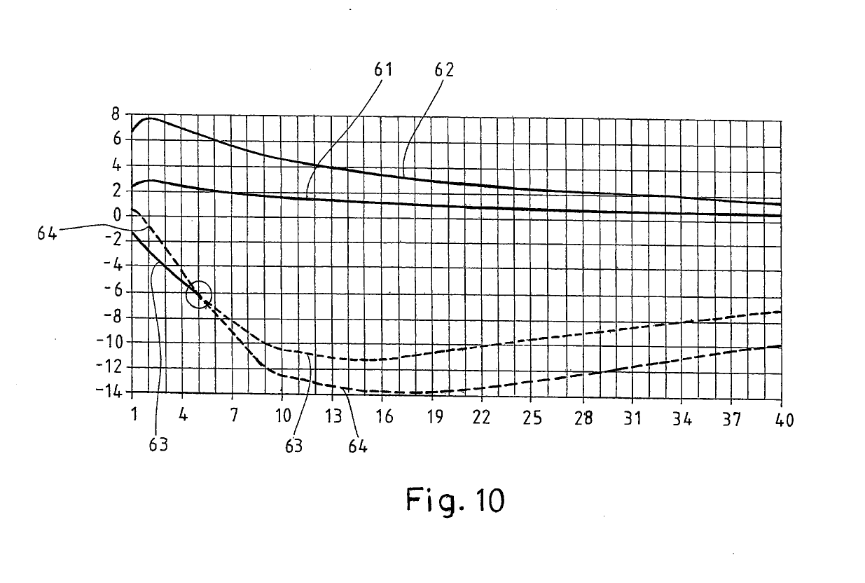

For this, FIG. 10 shows a diagram that illustrates the force over the permeability μᵣ of the tube 11. The ordinate shows the force F in Nm. The abscissa shows the permeability μᵣ. The curve 61 shows the holding force of the actuator in an end position when using permanent magnets with a remanence of 0.3 T. Reference number 63 shows by the dashed line the switching force of this actuator in the end position in the case of a coil linkage of 100 A/mm² and a remanence of the permanent magnet of 0.3 Tesla. Correspondingly, the curve 62 is the holding force of the actuator in the end position during use of permanent magnets with a remanence of 0.5 T and the curve 64 the switching force of the actuator in the end position in the case of a coil linkage of 100 A/mm² with a remanence of the permanent magnets or of the permanent magnet of 0.5 T. FIG. 10 thus shows the impact of the permeability of the tube 11 on the holding and switching forces of the bistable electromagnetic actuator. These curves were identified through an FEM simulation.

It can be seen that the holding forces increase up to a permeability of approx. 2 and then drop again and fall below the start value approximately at a permeability of 6. A greater effect is seen for the switching forces. For switching, the switching force must be negative. This results from the fact that the magnetic circuit around the coil is closed better through the permeability via the tube 11 and the magnetic flux generated by the coil is thereby increased. When using permanent magnets with 0.5 T, i.e. in the case of curves 61 and 63, it stands out that the actuator is not functional at a permeability of 1 since the switching force is positive. The switching force only becomes negative in the case of an increase in the permeability in the air gap. Both electromagnetic actuators reach the same switching force at the intersection of the curves 63 and 64, i.e. at a permeability of approximately 5. However, the holding force is almost three times as high at a remanence of 0.5 T. Up to a permeability of approximately 20, the electromagnetic actuator with a remanence of 0.5 T reaches a higher holding force than the absolute maximum of the holding force of the electromagnetic actuator with a remanence of 0.3 T. However, the switching force in this area is over four times as large.

With the help of the tube 11, which is alternatively referred to as the sliding element, the materials can be produced in a correspondingly machined manner, such as cold-formed. In particular, a roller-burnishing or deep-drawing is thereby considered. In particular, a cold-drawn tube can be used. Materials used in EMC shielding could also be used. This thereby concerns for example ferrites, such as for example nickel ferrites.

As an alternative, a plastic tube could be produced which is filled for example with ferromagnetic particles. Through the fill level of the plastic with ferromagnetic particles, the permeability of the tube can be set well. For example, permeabilities between 2 and 100 could be set without problems. During production, injected blanks can be machine-finished or an injection moulding production process can be used.

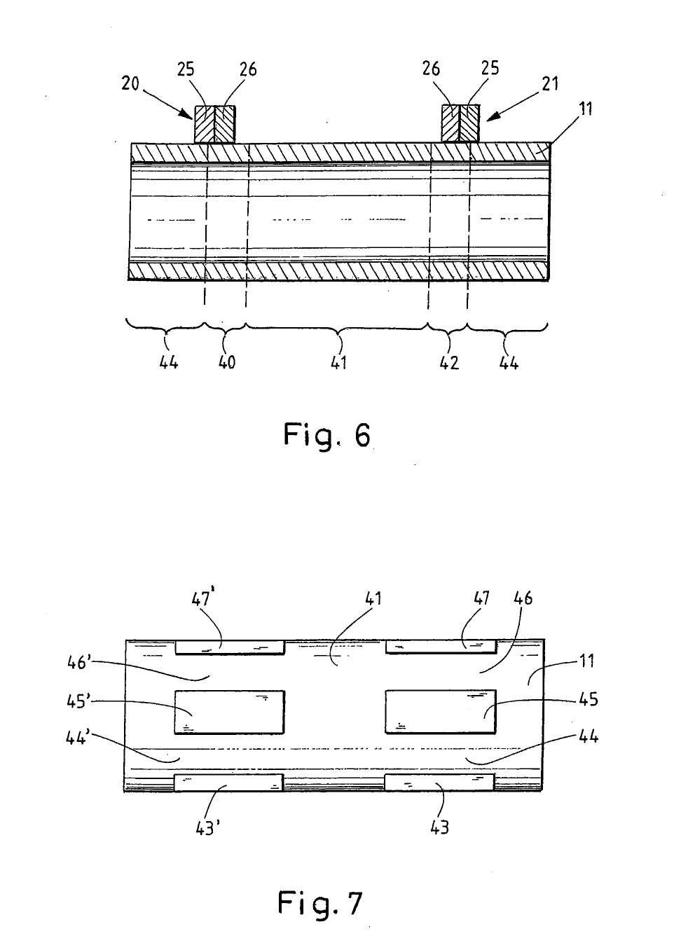

FIG. 6 shows a particular embodiment of a part of an actuator in a sectional representation, wherein in particular the tube 11 and a part of the magnets 20, 21 are shown, comprising correspondingly a magnetic south pole 25 and a magnetic north pole 26, in order to better represent the position in the tube 11. According to the embodiment in FIG. 4, the tube 11 is divided into different sections that are arranged behind each other longitudinally. Thus, the tube can be designed for example such that a middle area 41 is provided, which has for example a permeability of 1 or approximately 1. This middle area 41 is adjacent to two tube areas 40 and 42 which have an increased permeability of for example 2 to 100 or of 4 to 60 or of 6 to 40 or of 8 to 40 or another permeability in the range of 2 to 100. As indicated by the dashing, these tube areas 40 and 42 can lie in the area of the magnets 20 and 21 and can be slightly offset from these magnets. An end area 44 can then connect to both sides, in which the tube has a permeability of 1 or approximately 1. However, the end areas 44 can also have a correspondingly higher permeability and have in particular a permeability in the ranges 40 and 42. This embodiment prevents the magnetic flux from being lost through the tube between the magnets 20 and 21 for holding the movable element 10 and for switching the movable element 10. The magnetic flux is thereby correspondingly bundled by the tube 11, and namely in the radial direction through the tube 11.

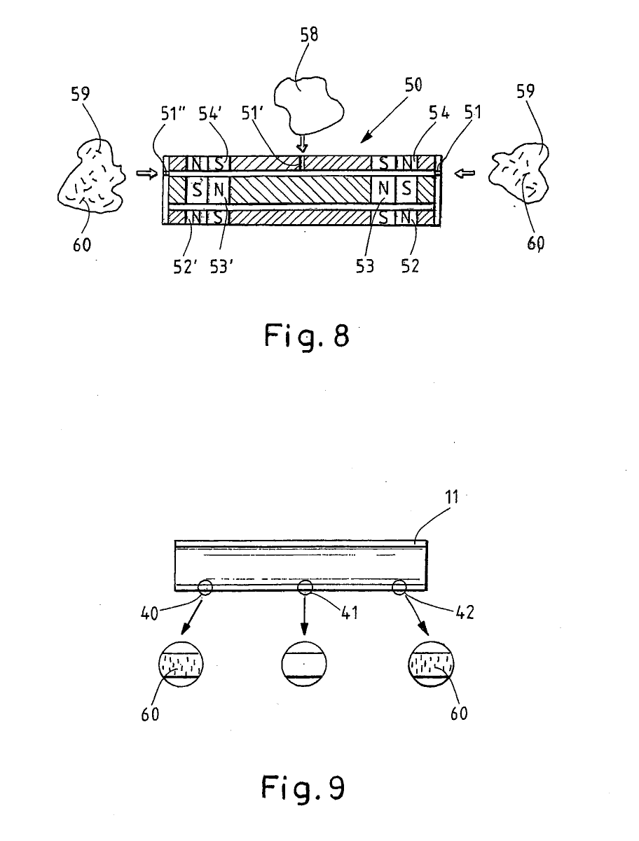

In order to produce a corresponding tube, for example an injection moulding can be used, in particular with a casting mould, as is shown in FIG. 8 in a schematic sectional representation. The casting mould 50 is shown here which has three openings 51, 5′ and 51″, which are used as gate marks. The casting mould 50 has an outer shell, an inner tube and covers on all sides. A hollow space which is tubular and from which the tube 11 is then formed, is designed between these elements. In the axial direction in the end areas of the casting mould 50, in particular slightly distanced from the front surfaces, corresponding magnets 52, 53 and 54 are provided on the right side and 52′, 53′ and 54′ on the left side, which can ensure a magnetization of ferromagnetic particles in a casting compound during the injection moulding. For example, the casting compound 59 with ferromagnetic particles 60 is introduced into the opening 51 and correspondingly a casting compound 59 with ferromagnetic particles 60 is also introduced into opening 51″. A casting compound 58, which in particular has no ferromagnetic particles, is introduced into opening 51′ into the middle. In this manner, a corresponding tube with different areas can be produced, which is shown schematically in FIG. 9. Here, the tube 11 is shown in a schematic sectional representation and corresponding areas 40, 41 and 42 below this tube are shown in enlarged detail. The aligned ferromagnetic particles 60 are shown in the areas left and right, i.e. in the enlarged details for areas 40 and 42, and the plastic not containing ferromagnetic particles is shown in the enlarged detail of area 41. This results in a very efficient production process.

The ferromagnetic particles 60 align themselves according to the field lines of the magnets 52, 53 and 54 or respectively 52′, 53′ and 54′ after introduction of the casting compounds 58 and 59. After the hardening of the casting compounds or respectively of the casting compound, which can for example be a plastic, such as for example a two-component polyester or epoxy resin, the corresponding permeability of the areas 40 and 42 is retained.

A further development of an actuator is given in that the tube 11, as shown schematically in a top view in FIG. 7, is divided in the circumferential direction in sections or respectively areas which have different permeabilities adjacently. This top view shows in the circumferential direction three areas provided with increased permeability and namely on the right side 43, 45 and 47 and two areas 44 and 46, which have a permeability of 1 or respectively approximately 1. Another area with increased permeability cannot be seen in FIG. 7 since it is covered. Correspondingly, in this exemplary embodiment, an area structuring is also provided with the areas 43′, 45′ and 47′ on the left side of the tube 11, which have an increased permeability, and 44′ and 46′, which have a permeability of approximately 1. The structuring of the areas in the circumferential directions serves to prevent the movable element from also experiencing a rotation during longitudinal movement. The movable element can then also have corresponding pole shoes 27 and 28 which are also structured in the circumferential direction of the movable element. These are then in magnetic engagement with the areas of the tube 11 structured in the circumferential direction.

Through the provision of magnets 52, 52′, 53, 53′, 54, 54′ in the casting mould 50, the ferromagnetic particles, which can be designed aspherically, in particular elongated, are aligned during the production of the tube 11. In the case of the produced sliding tube 11, an anisotropic permeability thereby results in the areas which existed in the effective range of the magnets of the casting mould 50 during production. It is thereby achieved that the sliding tube 11 in the actuator has sections, which permit a higher magnetic flux in the radial direction than in the axial direction. A magnetic short circuit of the magnetic fields, which lead through the magnets, comprising the areas 25 and 26, and through the pole shoes 23 and 23′, is thereby avoided or reduced. Through the alignment of the ferromagnetic particles, the susceptibility is thus increased in the radial direction of the magnetic flux compared to the axial direction.

The electromagnetic actuator can be used in endoscopes that have an optical system. In particular, a lens can be moved with the electromagnetic actuator such that it can be moved longitudinally along the longitudinal axis 35. A focussing or a movement of the focal length of the objective is thereby enabled. Instead of or in addition to the lens, a mirror can also be provided, by means of which the viewing direction of an operator in the distal area of the endoscope can be changed. Through the solution provided by the actuators disclosed herein, little construction effort with little space requirement is realizable so that the lumen available for example for lenses is reduced only slightly so that very bright objectives and thus also bright endoscopes are realizable.

All named characteristics, including those taken from the drawings alone and also individual characteristics which are disclosed in combination with other characteristics are considered alone and in combination as essential for the invention. Embodiments according to the invention can be realized by individual characteristics, or a combination of several characteristics.

### LIST OF REFERENCE NUMBERS

10 Movable element

11 Sliding tube

12 Fixed holding element

13 Lens

14 Lens

15 Lens

16 Stop

17 Stop

18 Distal end

19 Stator

20 Permanent magnet

21 Permanent magnet

22 1st intermediate part

23, 23′ 2nd intermediate part

24 Coil

25 Magnetic south pole

26 Magnetic north pole

27 Distal pole shoe

28 Proximal pole shoe

29 Distal stop element

30 Proximal stop element

31 Force

32 Magnetic flux

33 Magnetic flux

34 Moving force

35 Longitudinal axis

40, 41, 42 Tube area

43, 43′, 44, 44′ Tube area

45, 45′, 46, 46′ Tube area

47, 47′ Tube area

50 Casting mould

51, 51′, 51″ Opening

52, 52′ Magnet

53, 53′ Magnet

54, 54′ Magnet

58 Casting compound

59 Casting compound with ferromagnetic particles

60 Ferromagnetic particles

61 Holding force

62 Holding force

63 Switching force

64 Switching force

a Distance

b Distance

c Distance

What is claimed is:

1. An electromagnetic actuator for a medical instrument, wherein the electromagnetic actuator comprises:
a stator;
a movable element, which at least partially comprises one or more of a paramagnetic and ferromagnetic material and which is movable from a first position to a second position by the application of an electromagnetic field, and
a tube movably supporting the movable element in such a way that the movable element is longitudinally movable, wherein the tube comprises a ferromagnetic material.

2. The electromagnetic actuator according to claim 1, wherein the permeability of the tube lies at least in sections in a range, the lower limit of which is one of 1.2 or 2, 3, 4 or 5 and the upper limit of which is one of 200, 100, 40, 30, 25 or 12.

3. The electromagnetic actuator according to claim 1, wherein the tube contains a plastic filled with ferromagnetic particles.

4. The electromagnetic actuator according to claim 1, wherein the tube in the axial direction has areas, the permeability of which is different from each other.

5. The electromagnetic actuator according to claim 4, wherein at least one of the areas adjacent to a middle area of the tube has a higher permeability than the middle area.

6. The electromagnetic actuator according to claim 1, wherein at least one area of the tube has an anisotropic permeability.

7. The electromagnetic actuator according to claim 1, wherein the tube in the circumferential direction has areas, the permeability of which is different from an adjacent area in the circumferential direction.

8. The electromagnetic actuator according to claim 1, wherein the movable element can be held in the first position by a first permanent magnetic field and, after movement into the second position, can be held in the second position by a second permanent magnetic field.

9. The electromagnetic actuator according to claim 1, wherein the stator includes two permanent magnets which are oppositely poled.

10. The electromagnetic actuator according to claim 9, wherein the stator includes a coil is provided for generating the electromagnetic field.

11. The electromagnetic actuator according to claim 10, wherein the coil is arranged between the permanent magnets.

12. The electromagnetic actuator according to claim 1, further comprising first and second stops to define the first and the second positions, respectively.

13. The electromagnetic actuator according to claim 12, wherein, upon placement of the movable element at one of the first and second stops, a force acts in the direction of the one of the first and second stops on the movable element.

14. The electromagnetic actuator according to claim 9, further comprising one or more of a paramagnetic and/or ferromagnetic material arranged between the two permanent magnets of the stator.

15. The electromagnetic actuator according to claim 9, wherein the movable element, the coil, the tube and/or the two permanent magnets are annular in cross-section.

16. The electromagnetic actuator according to claim 1, wherein the medical instrument is an endoscope.

17. A medical instrument comprising the electromagnetic actuator according to claim 1.

18. The medical instrument according to claim 1, wherein the medical instrument is an endoscope.

19. A method for producing a tube, the method comprising:
arranging at least one magnet in a casting mould for casting the tube,
introducing a casting compound into the casting mould, wherein at least in an area of the casting mould, in which the at least one magnet is arranged, the casting compound includes ferromagnetic particles, and
hardening the casting compound to form the tube.
